(19) United States
(12) Patent Application Publication
(10) Pub. No.: US 2024/0180807 A1
(43) Pub. Date: Jun. 6, 2024

(54) HAIR GROWTH AGENT

(71) Applicant: ADJUVANT HOLDINGS CO., LTD., Kobe-shi, Hyogo (JP)

(72) Inventors: Hideki Takahashi, Kobe-shi (JP); Sota Nakamura, Kobe-shi (JP)

(73) Assignee: ADJUVANT HOLDINGS CO., LTD., Kobe-shi, Hyogo (JP)

(21) Appl. No.: 18/278,726

(22) PCT Filed: Feb. 25, 2022
(86) PCT No.: PCT/JP2022/008004

§ 371 Date: Aug. 24, 2023

(30) Foreign Application Priority Data
Feb. 26, 2021 (JP) 2021-029427

(51) Int. Cl. A61K 8/49; A61K 8/64; A61K 9/08; A61K 31/506; A61K 38/05; A61P 17/14; A61Q 7/00

(52) U.S. Cl. CPC A61K 8/4953; A61K 8/64; A61K 9/08; A61K 31/506; A61K 38/05; A61P 17/14; A61Q 7/00; A61K 2800/592

(57) ABSTRACT

PROBLEM: To provide a hair growth agent which is a topical agent that exhibits effect in terms of causing increase in hair shaft diameter and improving maximum hair shaft length and improving hair shaft elongation rate and new hair growth and increasing expression of genes contributing to hair growth in dermal papilla cells and promoting hair shaft growth at head hair, beard, eyelashes, and/or eyebrows.

SOLUTION MEANS: A hair growth agent which is a topical agent is made to contain active ingredient(s) in the form of palmitoyl dipeptide-5 diaminobutyloyl hydroxythreonine and palmitoyl dipeptide-5 diaminohydroxybutyrate, and/or minoxidil.

### TECHNICAL FIELD

The present invention relates to a hair growth agent. More particularly, it relates to a hair growth agent that contains palmitoyl dipeptide-5 diaminobutyloyl hydroxythreonine and palmitoyl dipeptide-5 diaminohydroxybutyrate, and/or minoxidil.

### BACKGROUND ART

There has been increasing demand for hair growth agents and other such topical agents that will improve hair type and/or hair quality and hair growth effect in mammals including humans. To improve hair type and/or hair quality and hair growth effect, active ingredients which contribute to regulation of the hair cycle, i.e., the hair life cycle, have been proposed and are in the process of coming onto the market in the form of hair growth agents.

For example, use of minoxidil as an active ingredient in a hair growth agent has been proposed (see Patent Reference Nos. 1 through 3 and so forth), and hair growth agents employing minoxidil as active ingredient have undergone clinical trials in humans and are on the market. However, for reasons such as the fact that the pharmaceutical use thereof within Japan is limited to male alopecia prematura, it has not adequately satisfied the broad needs of consumers who desire hair growth effect and hair type and/or hair quality improvement effect.

Palmitoyl dipeptide-5 diaminobutyloyl hydroxythreonine and palmitoyl dipeptide-5 diaminohydroxybutyrate are known as components in raw materials for cosmetics (see Patent Reference No. 4). However, there are no reports related to a hair growth effect of palmitoyl dipeptide-5 diaminobutyloyl hydroxythreonine and palmitoyl dipeptide-5 diaminohydroxybutyrate.

### PRIOR ART REFERENCES

### Patent References

Patent Reference No. 1: Specification of U.S. Pat. No. 4,139,619
Patent Reference No. 2: Japanese Patent Application Publication Kokai No. S63[1988]-150211
Patent Reference No. 3: Japanese Patent Application Publication Kokai No. S63[1988]-145217
Patent Reference No. 4: Japanese Patent No. 5028474

### SUMMARY OF INVENTION

### Problem to be Solved by Invention

It is an object of the present invention to provide a hair growth agent that possesses excellent hair growth action.

### Means for Solving Problem

As a result of intensive and repeated research for the purpose of solving the foregoing problems, the present inventor(s) discovered that use of palmitoyl dipeptide-5 diaminobutyloyl hydroxythreonine and palmitoyl dipeptide-5 diaminohydroxybutyrate as active ingredients permitted attainment of hair growth activity, and moreover that use of palmitoyl dipeptide-5 diaminobutyloyl hydroxythreonine and palmitoyl dipeptide-5 diaminohydroxybutyrate, and/or minoxidil, as active ingredient(s) permitted attainment of strong hair growth activity, which culminated in the present invention.

A first means in accordance with the present invention for solving the foregoing problems is a hair growth agent characterized in that it contains palmitoyl dipeptide-5 diaminobutyloyl hydroxythreonine and palmitoyl dipeptide-5 diaminohydroxybutyrate, and minoxidil, as active ingredients.

A second means in accordance with the present invention for solving the foregoing problems is a hair growth agent for use in combination with minoxidil, characterized in that it contains palmitoyl dipeptide-5 diaminobutyloyl hydroxythreonine and palmitoyl dipeptide-5 diaminohydroxybutyrate as active ingredients.

A third means in accordance with the present invention for solving the foregoing problems is a hair growth agent for use in combination with palmitoyl dipeptide-5 diaminobutyloyl hydroxythreonine and palmitoyl dipeptide-5 diaminohydroxybutyrate, characterized in that it contains minoxidil as active ingredient.

A fourth means in accordance with the present invention for solving the foregoing problems is the hair growth agent of any one among the first through third means in accordance with the present invention for use in causing new hair growth or hair shaft growth promotion.

A fifth means in accordance with the present invention for solving the foregoing problems is the hair growth agent of any one among the first through fourth means in accordance with the present invention used for causing improvement in hair shaft elongation rate.

A sixth means in accordance with the present invention for solving the foregoing problems is the hair growth agent of any one among the first through fourth means in accordance with the present invention used for causing improvement in maximum hair shaft length.

A seventh means in accordance with the present invention for solving the foregoing problems is the hair growth agent of any one among the first through fourth means in accordance with the present invention used for causing increase in hair shaft diameter.

An eighth means in accordance with the present invention for solving the foregoing problems is the hair growth agent of any one among the first through fourth means in accordance with the present invention used for causing increase in number of hairs.

A ninth means in accordance with the present invention for solving the foregoing problems is the hair growth agent of any one among the first through eighth means in accordance with the present invention in liquid solution form.

A tenth means in accordance with the present invention for solving the foregoing problems is the hair growth agent of any one among the first through ninth means in accordance with the present invention for use on head hair, beard, eyelashes, and/or eyebrows.

An eleventh means in accordance with the present invention for solving the foregoing problems is a method of manufacturing a hair growth agent, characterized by presence of an operation in which palmitoyl dipeptide-5 diaminobutyloyl hydroxythreonine and palmitoyl dipeptide-5 diaminohydroxybutyrate, and/or minoxidil, are made to be contained as active ingredient(s) in pharmaceutical preparation(s).

A twelfth means in accordance with the present invention for solving the foregoing problems is a method of manufacturing a hair growth agent for use in combination with minoxidil, characterized by presence of an operation in which palmitoyl dipeptide-5 diaminobutyloyl hydroxythreonine and palmitoyl dipeptide-5 diaminohydroxybutyrate are made to be contained as active ingredients in a pharmaceutical preparation.

A thirteenth means in accordance with the present invention for solving the foregoing problems is a method of manufacturing a hair growth agent for use in combination with palmitoyl dipeptide-5 diaminobutyloyl hydroxythreonine and palmitoyl dipeptide-5 diaminohydroxybutyrate, characterized by presence of an operation in which minoxidil is made to be contained as active ingredient in a pharmaceutical preparation.

A fourteenth means in accordance with the present invention for solving the foregoing problems is a hair growth agent kit that comprises a pharmaceutical preparation which contains minoxidil, and a pharmaceutical preparation which contains palmitoyl dipeptide-5 diaminobutyloyl hydroxythreonine and palmitoyl dipeptide-5 diaminohydroxybutyrate.

A fifteenth means in accordance with the present invention for solving the foregoing problems is a hair growth agent kit for use in combination with minoxidil, which comprises a pharmaceutical preparation that contains palmitoyl dipeptide-5 diaminobutyloyl hydroxythreonine and palmitoyl dipeptide-5 diaminohydroxybutyrate.

A sixteenth means in accordance with the present invention for solving the foregoing problems is a hair growth agent kit for use in combination with palmitoyl dipeptide-5 diaminobutyloyl hydroxythreonine and palmitoyl dipeptide-5 diaminohydroxybutyrate, which comprises a pharmaceutical preparation that contains minoxidil.

A seventeenth means in accordance with the present invention for solving the foregoing problems is a hair growth method characterized by administration of palmitoyl dipeptide-5 diaminobutyloyl hydroxythreonine and palmitoyl dipeptide-5 diaminohydroxybutyrate, and/or minoxidil, as active ingredient(s).

An eighteenth means in accordance with the present invention for solving the foregoing problems is a hair growth method comprising administering the hair growth agent of any one among the first through tenth means in accordance with the present invention to a subject.

Another means in accordance with the present invention for solving the foregoing problems is a scalp care agent that contains palmitoyl dipeptide-5 diaminobutyloyl hydroxythreonine and palmitoyl dipeptide-5 diaminohydroxybutyrate, and/or minoxidil, as active ingredient(s).

Moreover, another means in accordance with the present invention for solving the foregoing problems is a scalp symptom improvement method that comprises administering a scalp care agent which contains palmitoyl dipeptide-5 diaminobutyloyl hydroxythreonine and palmitoyl dipeptide-5 diaminohydroxybutyrate, and/or minoxidil, as active ingredient(s) to a subject.

### Benefit of Invention

By causing palmitoyl dipeptide-5 diaminobutyloyl hydroxythreonine and palmitoyl dipeptide-5 diaminohydroxybutyrate, and/or minoxidil, to be active ingredient(s) in a hair growth agent which is a topical agent, means in accordance with the present invention make it is possible to provide an excellent hair growth agent and scalp care agent that exhibit scalp care effect as well as effect in terms of causing increase in hair shaft diameter and improving maximum hair shaft length and improving hair shaft elongation rate and hair shaft growth promotion at head hair, beard, eyebrows, and/or eyelashes.

### BRIEF DESCRIPTION OF DRAWINGS

FIG. 1 contains graphs showing change in amount of genetic expression in human dermal papilla cells as a result of stimulation for 24 hours with mixtures of palmitoyl dipeptide-5 diaminobutyloyl hydroxythreonine and palmitoyl dipeptide-5 diaminohydroxybutyrate, and minoxidil; and with palmitoyl dipeptide-5 diaminobutyloyl hydroxythreonine and palmitoyl dipeptide-5 diaminohydroxybutyrate and minoxidil. (a) at FIG. 1 shows change in amount of genetic expression of FGF-7; (b) at FIG. 1 shows change in amount of genetic expression of VEGF.

FIG. 2 contains graphs showing change in amount of genetic expression in human dermal papilla cells as a result of stimulation for 72 hours with mixtures of palmitoyl dipeptide-5 diaminobutyloyl hydroxythreonine and palmitoyl dipeptide-5 diaminohydroxybutyrate, and minoxidil; and with palmitoyl dipeptide-5 diaminobutyloyl hydroxythreonine and palmitoyl dipeptide-5 diaminohydroxybutyrate and minoxidil. (a) at FIG. 2 shows change in amount of genetic expression of FGF-7; (b) at FIG. 2 shows change in amount of genetic expression of VEGF.

### EMBODIMENTS FOR CARRYING OUT INVENTION

Embodiments for carrying out the present invention are described below. Note that the present invention is not limited to these examples alone, it being of course possible to make any number of changes thereto without departing from the gist of the present invention.

The active ingredient(s) of a hair growth agent and a scalp care agent associated with the present invention comprise palmitoyl dipeptide-5 diaminobutyloyl hydroxythreonine (Palm-Lys-Val-Dab-Thr-OH) and palmitoyl dipeptide-5 diaminohydroxybutyrate (Palm-Lys-Val-Dab-OH), and/or minoxidil.

Concentrations of palmitoyl dipeptide-5 diaminobutyloyl hydroxythreonine and palmitoyl dipeptide-5 diaminohydroxybutyrate, and minoxidil, which constitute active ingredient(s) in a hair growth agent or scalp care agent in accordance with the present invention, are 0.001 wt % to 20 wt % of the entirety of the hair growth agent or scalp care agent. More specifically, they are 0.005 wt % to 10 wt %.

While hair growth agents and scalp care agents in accordance with the present invention may be used in the form of pharmaceutical preparations having any of a wide variety of modes of dosage forms serving as topical agents such as ointments, poultices, liniments, lotions, liquids for topical use, dusting powders, creams, gels, emulsions, hair tonics, hair sprays, microneedles, and so forth as cosmetics including cosmetics for the scalp and cosmetics for the eyelashes and/or eyebrows, beard, head hair, quasi-pharmaceutical agents, pharmaceutical agents, and so forth, there is no limitation with respect thereto.

Hair growth agents and scalp care agents in accordance with the present invention may be manufactured as a result of performance of operations which cause palmitoyl dipeptide-5 diaminobutyloyl hydroxythreonine and palmitoyl dipeptide-5 diaminohydroxybutyrate, and/or minoxidil, to be contained therein as active ingredient(s). Furthermore, in accordance with the present invention, hair growth agents and scalp care agents for use in combination with minoxidil may be manufactured as a result of performance of operations which cause palmitoyl dipeptide-5 diaminobutyloyl hydroxythreonine and palmitoyl dipeptide-5 diaminohydroxybutyrate to be contained as active ingredients therein; moreover, hair growth agents and scalp care agents for use in combination with palmitoyl dipeptide-5 diaminobutyloyl hydroxythreonine and palmitoyl dipeptide-5 diaminohydroxybutyrate may be manufactured as a result of performance of operations which cause minoxidil to be contained as active ingredient therein. Moreover, in addition to the foregoing operations, operations may be added which cause additive(s) to be contained therein for the purpose of formulation as pharmaceutical preparation(s).

In the context of hair growth agents and scalp care agents in accordance with the present invention, where palmitoyl dipeptide-5 diaminobutyloyl hydroxythreonine, palmitoyl dipeptide-5 diaminohydroxybutyrate, and/or minoxidil are to be contained in separate pharmaceutical preparations that will be provided, the dosage forms for those pharmaceutical preparations may be such that they are a combination of different dosage forms or the same dosage form, and they may also be provided in the form of a kit for providing pharmaceutical preparation(s) having such dosage form(s).

Furthermore, to the extent that it does not interfere with the hair growth effect and scalp care effect of the present invention, additives and/or other such components, presence of which would ordinarily be permitted in cosmetics including cosmetics for the scalp and cosmetics for the eyelashes and/or eyebrows, head hair, quasi-pharmaceutical agents, pharmaceutical agents, and so forth, may be additionally blended therein. As such additives and/or other such components, while excipients, stabilizers, corrigents, vehicle, dispersants, diluents, anionic surface active agents, amphoteric surface active agents, nonionic surface active agents, cationic surface active agents, anionic polymers, nonionic polymers, ethylene oxide-propylene oxide block copolymer, alcohols, emulsifiers, percutaneous absorption promoters, pH adjustors, preservatives, colorants, lipids, mineral oils, and other such oily components, moisturizing agents, thickeners, polymers, film-forming agents, ultraviolet light absorbers, cell activators, moisturizing agents, inorganic salts, functional beads and capsules, silicones, metal chelating agents, antioxidants, antiseptic agents, fresheners, deodorants, pigments, dyes, fragrances, sugars, amino acids, vitamins, organic acids, organic amines, plant extracts, clay minerals, various polymers, and other such viscosity modifiers, and so forth may be cited as examples, there is no limitation with respect thereto.

Hair growth agents and scalp care agents in accordance with the present invention may additionally contain known components having new hair growth effect, hair growth effect, hair tonic effect, and/or the like.

Administration dosage of active ingredient(s) per dose of a hair growth agent and scalp care agent of a means in accordance with the present invention may be adjusted so as to cause effect(s) of the hair growth agent and scalp care agent in accordance with the present invention to be exhibited. In addition, such administration dosage might for example be 0.005 mg to 200 mg, might more specifically be 0.05 mg to 100 mg, and might still more specifically be 0.5 mg to 10 mg.

So as to cause effect(s) of the hair growth agent and scalp care agent in accordance with the present invention to be exhibited, the number of administrations of a hair growth agent and scalp care agent in accordance with the present invention might be one administration or might be multiple administrations. In addition, the number of administrations of a hair growth agent and scalp care agent in accordance with the present invention might for example be 1 to 6 times per day. In addition, more specifically this might be 1 to 3 times per day, and still more specifically this might be 1 to 2 times per day.

Hair growth agents and scalp care agents in accordance with the present invention relate to hair shaft growth promotion, new hair growth, and hair loss prevention, and preferably relate to hair shaft growth promotion and new hair growth.

In the present specification, the term “hair shaft growth promotion” means improving hair shaft elongation rate, improving maximum hair shaft length, and/or increasing hair shaft diameter.

In the present specification, the term “new hair growth” means promoting growth of new hair and increasing number of hairs at follicle pores where new hair growth capability has been lowered or where new hair growth has stopped at a location where there is a small number of hairs or where there is no hair (no hair shaft extends to the exterior from the epidermis), and more specifically means shortening the telogen phase of the hair cycle and/or restarting a stopped hair cycle.

In the present specification, “to have hair shaft growth promotion effect” means acting in a way such as will be advantageous for promotion of hair shaft growth, and the quality by which hair shaft growth promotion effect is indicated is referred to as “hair shaft growth promotion activity”. Furthermore, “to have new hair growth effect” means acting in a way such as will be advantageous for new hair growth, and the quality by which new hair growth effect is indicated is referred to as “new hair growth promotion activity”.

In the present specification, the term “hair loss” means the phenomenon whereby the hair shaft comes free from the follicle pore, and more specifically means increase in inhibitory cytokines or the like which interfere with cell growth, and to cell death resulting therefrom. The quality by which hair loss prevention effect is indicated is referred to as “hair loss prevention activity”. Furthermore, “to have hair loss prevention effect,” which is a physiological phenomenon different from the qualities by which hair shaft growth promotion and/or new hair growth effect are indicated, means decreasing the number of hair shafts that come free from follicle pores as a result of reduction in or interference with inhibitory cytokines and suppression of cell death.

In the present specification, the term “scalp symptoms” means dandruff, roughness of the scalp, dryness of the scalp, erythema, itchiness, acne, and/or other such symptoms. In addition, in the present specification, the term “improvement of scalp symptoms” means improvement or suppression of dandruff, roughness of the scalp, dryness of the scalp, erythema, itchiness, acne, and/or the like.

A hair growth agent in accordance with the present invention may be used to improve hair shaft elongation rate and/or maximum hair shaft length. In addition, with respect to hair shaft elongation rate, as compared with hair shaft elongation rate pursuant to hair cycle reference data, it may for example cause a maximum improvement of on the order of 110%, more specifically it may cause improvement on the order of 25% to 110%, and still more specifically it may cause improvement on the order of 33% to 110%. Furthermore, with respect to maximum hair shaft length, as compared with maximum hair shaft length pursuant to hair cycle reference data, it may for example cause a maximum improvement of on the order of 49%, more specifically it may cause improvement on the order of 1% to 49%, and still more specifically it may cause improvement on the order of 2% to 49%.

A hair growth agent in accordance with the present invention may be used to increase hair shaft diameter.

A hair growth agent in accordance with the present invention may be used to promote growth of new hair and increase the number of hairs at follicle pores where new hair growth capability has been lowered or where new hair growth has stopped at a location where there is a small number of hairs or where there is no hair (no hair shaft extends to the exterior from the epidermis), and more specifically may be used to shorten the telogen phase of the hair cycle and/or restart a stopped hair cycle.

Hair growth agents and scalp care agents in accordance with the present invention may be used not only for humans but also for domesticated animals, animal pets, and/or other such animals. One aspect of the present invention provides a scalp symptom improvement method and/or a hair growth method that includes administration of topical agent(s) which contain palmitoyl dipeptide-5 diaminobutyloyl hydroxythreonine and palmitoyl dipeptide-5 diaminohydroxybutyrate, and/or minoxidil, to subject(s) which may include human(s), domesticated animal(s), animal pet(s), and/or other such animal(s).

### Working Examples

Exemplary Test 1: Evaluation of Human Dermal Papilla Cell FGF-7 Gene and VEGF Gene Expression

The FGF-7 gene and the VEGF gene are expressed in dermal papilla cells, and contribute to expression of such effects as increase in hair shaft diameter and improving maximum hair shaft length and improving hair shaft elongation rate and hair shaft growth promotion at head hair, beard, eyebrows, and/or eyelashes. Human dermal papilla cells were therefore used, and evaluation was carried out with respect to increase in FGF-7 gene and VEGF gene expression for the respective components.

### 1. Materials and Methods

### (1) Human Dermal Papilla Cells and Culture Medium

Human dermal papilla cells (Catalog No. CA60205a; Caucasian; derived from 29-year-old male; Toyobo Co., Ltd. (Japan)) were purchased, testing and evaluation being carried out with maintenance and culture of cells being performed as described in the protocol.

### (2) Drugs

As drugs for testing, drug solutions of the following respective concentrations (final concentrations) were prepared and used.
Comparative Example 1: 30 μM minoxidil
Working Example 1: 0.025% solution of mixture of palmitoyl dipeptide-5 diaminobutyloyl hydroxythreonine and palmitoyl dipeptide-5 diaminohydroxybutyrate
Working Example 2: 0.1% solution of mixture of palmitoyl dipeptide-5 diaminobutyloyl hydroxythreonine and palmitoyl dipeptide-5 diaminohydroxybutyrate
Working Example 3: 3% solution of mixture of palmitoyl dipeptide-5 diaminobutyloyl hydroxythreonine and palmitoyl dipeptide-5 diaminohydroxybutyrate
Working Example 4: Liquid mixture of 30 μM minoxidil, and 0.1% solution of mixture of palmitoyl dipeptide-5 diaminobutyloyl hydroxythreonine and palmitoyl dipeptide-5 diaminohydroxybutyrate
Working Example 5: Liquid mixture of 30 μM minoxidil, and 3% solution of mixture of palmitoyl dipeptide-5 diaminobutyloyl hydroxythreonine and palmitoyl dipeptide-5 diaminohydroxybutyrate

### (3) Test Procedure

A 96-well plate was seeded with human dermal papilla cells so as to obtain 5×10⁴ thereof per well. Following culture for 1 day within a CO₂ incubator (5% CO₂; 37° C.), the culture medium was replaced with culture medium which contained the respective drugs for testing. The cell plate was thereafter returned to the CO₂ incubator, and this was further cultured for 24 hours or 72 hours. Following culture, total RNA was extracted from the respective wells and was recovered, and this was reverse-transcribed into cDNA. The cDNA that was prepared was used to respectively measure FGF-7 gene and VEGF gene expression in accordance with the real-time PCR method. The GAPDH gene was used as an internal standard, the amounts of FGF-7 gene and VEGF gene expression being calculated relative to the negative control group.

A FastGene RNA Basic Kit (Catalog No. FG-80250; Nippon Genetics Co., Ltd. (Japan)) was used to recover total RNA from cells.

100 μL of lysis buffer RL was added thereto per well, and the cells were lysed by pipetting. 100 μL of 70% ethanol was added to the cell lysate, and this was mixed by pipetting. The sample solution was added to a FastGene RNA binding column, and this was centrifuged at room temperature for 1 minute at 10000 g. The filtrate that passed through the column was discarded from the collection tube, and after returning the FastGene RNA binding column to its original collection tube, 600 μL of wash buffer RW1 was added to the FastGene RNA binding column, and this was centrifuged at room temperature for 1 minute at 10000 g. The FastGene RNA binding column was transferred to a new collection tube that was placed thereat, 700 μL of wash buffer RW2 was added to the FastGene RNA binding column, and this was centrifuged at room temperature for 1 minute at 10000 g. The FastGene RNA binding column was transferred to a new collection tube that was placed thereat, and this was centrifuged at room temperature for 1 minute at 15000 g. The FastGene RNA binding column was transferred to a new collection tube that was placed thereat, 50 μL of elution buffer RE was added at the center of the membrane of the FastGene RNA binding column, and this was centrifuged at room temperature for 1 minute at 10000 g to recover the purified RNA. Concentration of the recovered RNA was measured using a NanoDrop Lite (Catalog No. ND-LITE; Thermo Fisher Scientific K.K.), and this was stored at −80° C. until the following cDNA creation procedure.

A FastGene scriptase II cDNA synthesis 5X Ready Mix (Catalog No. NE-LS64; Nippon Genetics Co., Ltd. (Japan)) was used to synthesize cDNA. Dilution with RNase-free Water was carried out so as to cause concentration of total RNA produced in a new tube to be 20 ng/mL, 4 μL of FastGene scriptase II cDNA synthesis 5X Ready Mix was added to 16 μL of this sample solution, and this was agitated by vortexing. A MiniAmp thermal cycler (Thermo Fisher Scientific K.K.) was used to incubate this at 25° C. for 10 minutes, 42° C. for 60 minutes, and 85° C. for 5 minutes to synthesize cDNA.

The cDNA that was synthesized in accordance with the foregoing method was used to carry out real-time PCR. At prescribed wells in a 96-well plate, respective dilute solutions of cDNA template were added, Thunderbird SYBR qPCR Mix (Catalog No. QPS-201; Toyobo Co., Ltd. (Japan)) and primer were added thereto and mixed therewith, and gene expression was analyzed using a QuantStudio 7 Flex Real-Time PCR System (Catalog No. 4485693; Thermo Fisher Scientific K.K.). The PCR reaction was such that 40 cycles of 95° C. for 5 seconds, 60° C. for 30 seconds, 72° C. for 30 seconds were carried out.

Primers specific for the GAPDH gene which was used as internal standard, and primers specific for the VEGF gene and primers specific for the FGF-7 gene, these having been used for testing, are indicated below.
Primers for detecting FGF-7 gene expression
   Forward: tctgtcgaacacagtggtacctgag (Sequence No. 1)
   Reverse: gccactgtcctgatttccatga (Sequence No. 2)
Primers for detecting VEGF gene expression
   Forward: atcttcaagccatcctgtgtgc (Sequence No. 3)
   Reverse: caaggcccacagggallllc (Sequence No. 4)
Primers for detecting GAPDH gene expression
   Forward: catccctgcctctactggcgctgcc (Sequence No. 5)
   Reverse: ccaggatgcccttgagggggccctc (Sequence No. 6)

Relative amounts of expression of the respective genes were calculated as follows.

For each gene, Ct value (number of PCR cycles) was calculated based on the intersection of the amplification curve with the threshold line. The relative amount of expression is the target gene Ct value less the internal standard GAPDH gene Ct value.

### 2. Results

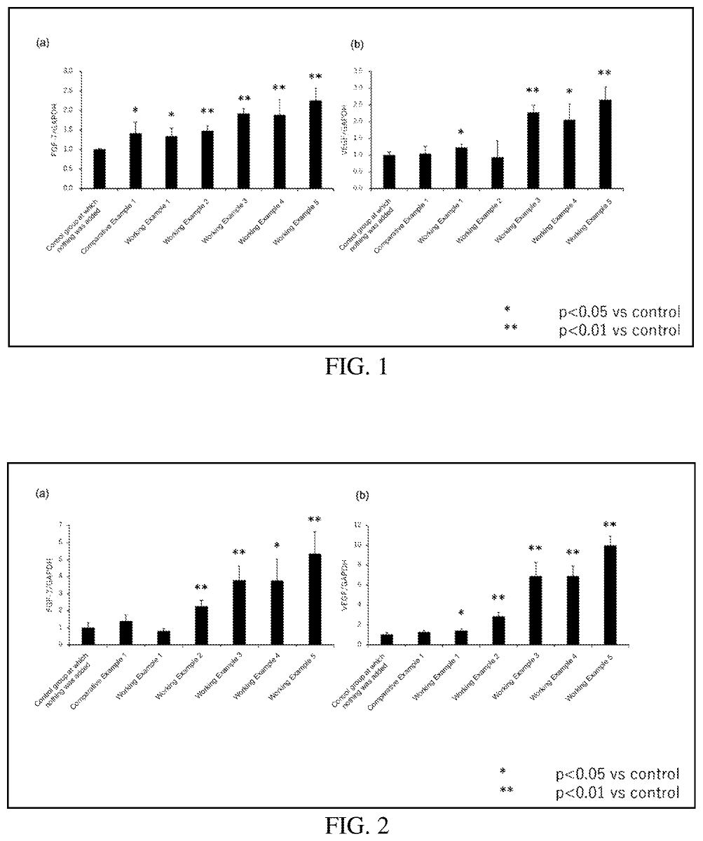

Change in amount of expression of the FGF-7 gene and the VEGF gene following action for 24 hours and 72 hours by mixtures of palmitoyl dipeptide-5 diaminobutyloyl hydroxythreonine and palmitoyl dipeptide-5 diaminohydroxybutyrate on human dermal papilla cells was measured, the results thereof being respectively shown in FIG. 1 (24 hours) and FIG. 2 (72 hours).

As shown in FIG. 1, it was found that causing a mixture of palmitoyl dipeptide-5 diaminobutyloyl hydroxythreonine and palmitoyl dipeptide-5 diaminohydroxybutyrate (Working Examples 1 through 3) to act for 24 hours on human dermal papilla cells resulted in increases in the amount of expression of the FGF-7 gene and the amount of expression of the VEGF gene as compared with the control group at which nothing had been added. In addition, it was found that increasing the amount of a mixture of palmitoyl dipeptide-5 diaminobutyloyl hydroxythreonine and palmitoyl dipeptide-5 diaminohydroxybutyrate that was added thereto caused the amount of expression of the FGF-7 gene and the amount of expression of the VEGF gene to be greater than that which was produced as a result of action of minoxidil (Comparative Example 1).

Moreover, it was found that causing a liquid mixture of minoxidil and a mixture of palmitoyl dipeptide-5 diaminobutyloyl hydroxythreonine and palmitoyl dipeptide-5 diaminohydroxybutyrate (Working Examples 4 through 5) to act for 24 hours on human dermal papilla cells resulted in increase in the amount of expression of the FGF-7 gene and the amount of expression of the VEGF gene that was more marked than was the case with minoxidil alone or with only a mixture of palmitoyl dipeptide-5 diaminobutyloyl hydroxythreonine and palmitoyl dipeptide-5 diaminohydroxybutyrate.

It was thus found that a mixture of palmitoyl dipeptide-5 diaminobutyloyl hydroxythreonine and palmitoyl dipeptide-5 diaminohydroxybutyrate was useful, in dermal papilla cells, as an active ingredient in a hair growth agent exhibiting effects in terms of causing increase in expression of the FGF-7 gene and expression of the VEGF gene, and causing increase in hair shaft diameter and improving maximum hair shaft length and improving hair shaft elongation rate and hair shaft growth promotion at head hair, beard, eyelashes, and/or eyebrows.

Moreover, it was found that use of minoxidil in combination with a mixture of palmitoyl dipeptide-5 diaminobutyloyl hydroxythreonine and palmitoyl dipeptide-5 diaminohydroxybutyrate caused expression of the FGF-7 gene and expression of the VEGF gene in dermal papilla cells to be such that—as compared with the situation when either a mixture of palmitoyl dipeptide-5 diaminobutyloyl hydroxythreonine and palmitoyl dipeptide-5 diaminohydroxybutyrate, or minoxidil, served as active ingredient(s)—there was more significant increase in expression of the FGF-7 gene and expression of the VEGF gene in dermal papilla cells, and that these were useful as active ingredients in a hair growth agent exhibiting effects in terms of causing increase in hair shaft diameter and improving maximum hair shaft length and improving hair shaft elongation rate and hair shaft growth promotion at head hair, beard, eyelashes, and/or eyebrows.

### INDUSTRIAL UTILITY

As a result of using palmitoyl dipeptide-5 diaminobutyloyl hydroxythreonine and palmitoyl dipeptide-5 diaminohydroxybutyrate, and/or minoxidil, as active ingredient(s) in a hair growth agent, a means in accordance with the present invention makes it possible to provide a novel scalp care agent and hair growth agent that exhibit scalp care effect as well as effect in terms of improving maximum hair shaft length and effect in terms of improving hair shaft elongation rate and hair shaft growth promotion effect at head hair, beard, eyelashes and/or eyebrows, and/or other such hair.

1. A hair growth agent characterized in that it contains palmitoyl dipeptide-5 diaminobutyloyl hydroxythreonine and palmitoyl dipeptide-5 diaminohydroxybutyrate, and minoxidil, as active ingredients.

2. A hair growth agent for use in combination with minoxidil, characterized in that it contains palmitoyl dipeptide-5 diaminobutyloyl hydroxythreonine and palmitoyl dipeptide-5 diaminohydroxybutyrate as active ingredients.

3. A hair growth agent for use in combination with palmitoyl dipeptide-5 diaminobutyloyl hydroxythreonine and palmitoyl dipeptide-5 diaminohydroxybutyrate, characterized in that it contains minoxidil as active ingredient.

4. The hair growth agent according to claim 1 for use in causing new hair growth or hair shaft growth promotion.

5. The hair growth agent according to claim 1 used for causing improvement in hair shaft elongation rate.

6. The hair growth agent according to claim 1 used for causing improvement in maximum hair shaft length.

7. The hair growth agent according to claim 1 used for causing increase in hair shaft diameter.

8. The hair growth agent according to claim 1 used for causing increase in number of hairs.

9. The hair growth agent according to claim 1 in liquid solution form.

10. The hair growth agent according to claim 1 for use on head hair, beard, eyelashes, and/or eyebrows.

11-17. (canceled)

18. A hair growth method comprising administering the hair growth agent according to claim 1 to a subject.
